SEQUENCE LISTING (1 sequence)

SEQ ID NO: 1 - protein, 38 aa, Homo sapiens
  PEPTIDE (1)..(38)
  ref <301>: AUTHORS: Hongmin W., Lim P., Karbowski M., Monteiro M.
  ref <302>: TITLE: Effects of overexpression of Huntingtin proteins on mitochondrial integrity
  ref <303>: JOURNAL: Human Molecular Genetics
  ref <304>: VOLUME: 18
  ref <305>: ISSUE: 4
  ref <306>: PAGES: 737-752
  ref <307>: DATE: 2009-02-15
  ref <313>: RELEVANT RESIDUES IN SEQ ID NO: (1)..(38)
QQQQQQQQQQQQQQQQQQQQQQQQQQQQQQQQQQQQQQ

(19) United States
(12) Patent Application Publication
(10) Pub. No.: US 2015/0353960 A1
(43) Pub. Date: Dec. 10, 2015

(54) EFFICIENT PRODUCTION OF BIOFUELS FROM CELLS CARRYING A METABOLIC-BYPASS GENE CASSETTE

(71) Applicant: Ebio, LLC, Houston, TX (US)

(72) Inventors: Trent Nguyen, Houston, TX (US); Martin J. Van Sickels, Spring, TX (US)

(73) Assignee: Ebio, LLC, Houston, TX (US)

(21) Appl. No.: 14/737,261

(22) Filed: Jun. 11, 2015

Related U.S. Application Data
(63) Continuation-in-part of application No. 13/694,542, filed on Dec. 12, 2012.
(60) Provisional application No. 61/570,730, filed on Dec. 14, 2011.

(51) Int. Cl. C12P 7/06; B01D 61/36; B01D 3/00; B01D 3/40

(52) U.S. Cl. CPC C12P 7/065; B01D 3/40; B01D 61/364; B01D 3/001

(57) ABSTRACT

Increasing the production of a glycolytic intermediate and the production of an organic compound, such as ethanol, by a bacteria expressing a polyglutamine protein is achieved by preparing a feed stream; combining the bacteria and feed stream; fermenting the feed stream; cooling the feed stream during fermentation; recovering the organic compound; and concentrating the organic compound. The feed stream can be a waste stream from an ethanol production facility. The process can allow for a similar yield to the ethanol plant alone but with the use of less feed stock, such as corn, sugarcane, sorghum, cassava, switchgrass, and wood chips.

### RELATED APPLICATIONS

This application claims priority to U.S. patent application Ser. No. 13/694,542 filed on Dec. 12, 2012 which is specifically incorporated by reference in its entirety herein. This application also claims the benefit of U.S. Provisional Patent Application. 61/570,730, filed Dec. 14, 2011 which is specifically incorporated by reference in its entirety herein.

### FIELD

The disclosure relates generally to production of organic compounds. The disclosure relates specifically to ethanol production. The disclosure relates to a process for increasing the production of a glycolytic intermediate and/or an organic compound as defined herein by a cell that is able to express a nucleic acid molecule, wherein the expression of the nucleic acid molecule gives the cell the ability to increase the production of a glycolytic intermediate such as pyruvate or glyceraldehyde 3-phosphate to produce said organic compound. Further, the disclosure relates to an Escherichia coli cell for use in this process that is able to use protein and/or sugar as a carbon source.

### BACKGROUND

The energy-producing organisms such as plants, some algae, and cyanobacteria produce energy not only for themselves but also for almost all other organisms on Earth. The product of photosynthesis is glucose, the immediate energy source of cells. Once photosynthesized, glucose can be used either to make energy for cellular work or converted into complex molecules of starch, oil, and proteins as stored energy, structural components, or molecular machineries. Proteins in cells serve critical functions such as structural components, enzymes, transport proteins, and cellular energy source. The structural components of the plant cell wall are made up of 2-10% proteins, and the cell membrane contains 50% proteins. The breakdown of proteins and mobilization of amino acids depend on the physiologic needs of plants under various conditions such as oxidative stress, salinity, seasonal change, and developmental stages.

Plants grown in a medium with high salt concentrations have lower energy levels, are shorter, have smaller biomass, and have a lower protein content in comparison with plants grown in low salt concentrations (Maria et al. 2001). As a case in point, Asish et al. (2005) showed that a salt-sensitive protein SSP-23 degrades at high salt concentration in the mangrove plant species Bruguiera parviflora, and the phenomenon is suggestive of a salt tolerance mechanism, which adjusts for the osmolarity difference by releasing the protein's amino acid content. High-salt medium seems to be a catalyst for protein breakdown like the SSP-23 degradation and its amino acid mobilization.

Plants adjust the level of energy utilization according to their developmental needs. During the winter, energy is reserved in the form of starch, proteins, and amino acids, which are derived from glucose photosynthesized during late summer. In the fresh pith of tobacco plant species Nicotiana tabacum, which represents a slow-growing tissue much like plants in winter months, 88% of the total cellular amino acids are present in a soluble pool with only 12% incorporated into proteins. Conversely the plant's callus, a rapid outgrowth tissue from growing the pith tissue in an artificial nutrient-rich medium, has only 8% of total amino acids in the soluble pool and 92% in proteins. Further, the most abundant amino acids in the soluble pool are glutamine, asparagine, glutamic acid, and aspartic acid (Kemp et al. 1972). The amino acid reserve of alanine, arginine, and asparagine comes from the nitrogen fixation of ammonia, probably via glutamine (Menegus et al., 1993). These reserve sources of energy and nitrogen-rich compounds are tapped in the spring when apical growth begins.

During the metabolically-dormant winter, plants do not produce much photosynthesized oxygen that can breakdown cell membrane and, thus, release its protein content. For example, some rhizomes (creeping rootstalks) such as P. australis, A. calamus, and S. lacustris keep their membranes intact during the winter. The growth condition that begins in the spring provides a sudden surge in oxygen that can damage cell membrane though the process called lipid peroxidation. Then, membrane-bound proteins are released, which provide an amino acid pool as a source for both energy and for making other proteins needed for growth. However, the rootstalk plant species Iris pseudocorus produces the anti-oxidant enzyme superoxide dismutase (SOD) during the anoxic winter season for stabilizing the oxidant surge in the spring. For those species that do not produce SOD just before spring, the change in season provides a burst of protein pool as a source of energy for the growth requirement.

Under the oxygen deprivation condition called anoxia, plants arrest oxidative phosphorylation and produce only 2 molecules of ATP during fermentation rather 36 ATP under oxygen-using metabolism. In response to anoxic stress, plants adapt by increasing the ATP production rate (Pasteur Effect) although not totally making up for the energy deficit. Under anoxia, the synthesis of plant proteins is inhibited by the destabilization of the protein-producing machineries of polysomes (Baily-Serres, 1990). Some genes turned on by anoxia are metabolism-specific and include alcohol dehydrogenase, pyruvate decarboxylase, enolase, glucose-6-phosphate isomerase, glyceraldehyde-3-phosphate dehydrogenase, lactate dehydrogenase, and sucrose synthase (Sachs et al. 1996), and these enzymes speed up the conversion of energy substrates into energy molecules to compensate for the lack of an energetic state during anoxia. With limited protein synthesis during anoxia, growth is impeded in most plant tissues except for the seed leaf (coleoptile) of germinating rice seedlings, which is unique in its fast growing rate under anoxia while other organs of the seedlings are inhibited from growing (Vartapetian et al., 1978). The energy for the coleoptile growth may come from the rice seed covering layer aleurone, which increases its soluble protein pool in addition to its protease concentrations during germination (Miyuki et al., 2002). With the intact rice seed containing 7-9% protein content, the significant pool of amino acids can provide the initial substrates for conversion into sugar by gluconeogenesis.

The growth-inhibiting stress factors mentioned above ultimately deprive plants' ability to make new sources of energy by inhibiting access to either water, sunlight, or oxygen, all necessary components of photosynthesis. If the immediate energy source is not supplied, plants resort to stored sources of energy locked in starch, oil, and proteins as seen under stress conditions. All 18 of the 20 naturally-occurring amino acids are precursors of substrates for gluconeogenesis to make glucose. If plants were engineered to be more efficient at performing gluconeogenesis, specific substrates along the metabolic pathways should be targeted for manipulation. Pyruvate, a substrate situated at a metabolic crossroad, can feed it into fermentation under anaerobic condition or cell respiration under aerobic condition. To divert pyruvate from entering anaerobic or aerobic respiration and, thus, depleting a viable source for glucose synthesis, pyruvate can be induce-metabolized into oxaloacetate by the over production of pyruvate carboxylase, the enzyme for the conversion. The abundance of oxaloacetate, a precursor for gluconeogenesis, may push the reactions forward. Pyruvate-oxaloacetate conversion also may stimulate the conversions of alanine, cysteine, glycine, serine, and threonine amino acids into pyruvate. The remaining 13 amino acids in FIG. 4 also may be stimulated to enter the cycle for the production of oxaloacetate if the accumulated substrate pool were used for phosphoenolpyruvate synthesis by PEPCK (phosphoenolpyruvate carboxylase).

When plants are subjected to various stress and growth-limiting conditions, metabolism is switched from energy storing (anabolism) to energy usage (catabolism). During periods of energy abundance, plants have evolved to store the excess energy in starch, oil, and proteins. When energy input is limited, the stored sources of energy can undergo metabolic interconversion into glucose via gluconeogenesis. Proteins are a major category of biomolecules in plants (corn kernels contain 9-10% proteins), and tapping into methods to convert proteins to sugars provide an added source of accessible substrates for ethanol production. Stress factors can mobilize the stored energy, and plants also can be engineered to actively take the gluconeogenic pathway. However, biomass buildup depends on the constant energy input without excessive external stress. To harvest the most possible sugars, plants can be allowed to build up biomass under nutrient-rich conditions then be subjected to stress just before harvesting, or plants can be engineered to grow normally while metabolizing sugars. How much economically-viable products we can extract from algal biomass depend on the available forms. A major hurdle in biofuel production is yield, and efforts to increase yield has focused on genetic engineering.

The current standard ethanol fermentation uses glucose sugar as feedstock, thus, limiting yield. The U.S. ethanol industry uses mostly corn as a feedstock, which contains 72% starch sugar that must be processed into glucose sugar for ethanol fermentation. Another source of glucose comes from cellulosic materials, which also have to be processed into glucose. Therefore, there still is a need for an alternative and improved production process of ethanol, which does not have all the drawbacks of existing processes. The engineered organisms herein described can use proteins in addition to sugars as carbon sources, therefore, increasing ethanol fermentation yield. Although the sugar glucose is the immediate energy source for cells, they can convert other nutrients like proteins, fats, and carbohydrates into glucose by the metabolic process called gluconeogenesis. The engineered organisms as described herein have enhanced gluconeogenesis pathways that can build up glucose, the feedstock for ethanol fermentation. This system has several advantages over the current ethanol fermentation system including increasing ethanol yield, using proteins and carbohydrates as sources of carbon, and making the clean biofuel production more economical. A process is needed that converts waste stream into ethanol.

### SUMMARY

An embodiment of the disclosure is a process for increasing the production of a glycolytic intermediate and the production of an organic compound by bacteria comprising preparing a feed stream; combining the bacteria and feed stream; fermenting the feed stream; cooling the feed stream during fermentation; recovering the organic compound; and concentrating the organic compound; wherein the bacteria comprises a nucleic acid molecule comprising a CAG/CAA repeat that encodes a polyglutamine protein that increases production of a glycolytic intermediate; and wherein a starting substrate is selected from at least one of the group consisting of sugar and protein. In an embodiment, the organic compound is selected from the group consisting of ethanol, butanol, ethylene, 1,3-propanediol, propanol, D-lactate, acetone, and fatty acid. In another embodiment, the glycolytic intermediate is pyruvate. In an embodiment, the bacteria is Escherichia coli. In another embodiment, the feed stream comprises yeast cells or portions thereof. In an embodiment, the feed stream is a waste stream. In another embodiment, the waste stream is produced from one selected from the group consisting of corn, sugarcane, sorghum, cassava, switchgrass, and wood chips. In an embodiment, the waste stream is produced from one selected from the group consisting of beer, wine, bio-ethanol manufacturing, and bio-butanol manufacturing. In another embodiment, the polyglutamine protein is that of SEQ ID NO: 1. In yet another embodiment, preparing the feed stream comprises lowering the temperature of the feed stream. In an embodiment, recovering the organic compound comprises utilizing one method selected from the group consisting of distillation, membrane separation, and extractive distillation. In an embodiment, the process further comprises concentrating the organic compound by at least one column. In another embodiment, the process further comprises removing at least some of the water from the ethanol. In an embodiment, removing at least some of the water from the ethanol is performed using a molecular sieve drying system. In another embodiment, the nucleic acid molecule comprising a CAG/CAA repeat that encodes a polyglutamine protein is integrated into the genome of the bacteria. In yet another embodiment, the nucleic acid molecule comprises a CAG/CAA repeat that encodes a polyglutamine protein is present on a plasmid. In yet another embodiment, the organic compound comprises about 94.5% ethanol. In an embodiment, fermenting the feed stream occurs at a positive pressure. In an embodiment, the positive pressure is between about 20 psia and 30 psia. In an embodiment, the temperature during fermenting the feed stream is about 95° F.

The foregoing has outlined rather broadly the features of the present disclosure in order that the detailed description that follows can be better understood. Additional features and advantages of the disclosure will be described hereinafter, which form the subject of the claims.

### BRIEF DESCRIPTION OF THE DRAWINGS

In order that the manner in which the above-recited and other enhancements and objects of the disclosure are obtained, a more particular description of the disclosure briefly described above will be rendered by reference to specific embodiments thereof which are illustrated in the appended drawings. Understanding that these drawings depict only typical embodiments of the disclosure and are therefore not to be considered limiting of its scope, the disclosure will be described with additional specificity and detail through the use of the accompanying drawings in which:

FIG. 1 depicts cellular respiration and ethanol fermentation. The engineered cells have inhibited cell respiration but enhanced ethanol fermentation from pyruvate accumulation.

FIG. 2 depicts the Electron Transport Chain. Polyglutamine inhibits oxidative phosphorylation of ATP from electron transport reactions.

FIG. 3 depicts construction of a recombinant strain. CAG/CAA repeat sequence was synthesized. The CAG/CAA repeat DNA is linked to a promoter, which drives its expression. Homologous combination incorporates CAG/CAA repeat into the cell's genome.

FIG. 4 depicts gluconeogenesis. Amino acids are carbon sources that can be converted into glucose by cells such as Escherichia coli.

FIG. 5 depicts basic PFD prep and fermentation (PFD-1).

FIG. 6 depicts basic PFD beer still and rectifier (PFD-2).

FIGS. 7A, 7B, and 7C depict a mass balanced summary.

FIG. 8 depicts use of various components being of use to perform the Alcoli™ process, including the Alcoli™ agent, new agents, new plants, and plant retrofits.

FIG. 9 depicts a flowchart the interaction between the production of ethanol from corn and utilization of the waste stream in the Alcoli™ process to produce ethanol.

### DETAILED DESCRIPTION

The particulars shown herein are by way of example and for purposes of illustrative discussion of the preferred embodiments of the present disclosure only and are presented in the cause of providing what is believed to be the most useful and readily understood description of the principles and conceptual aspects of various embodiments of the disclosure. In this regard, no attempt is made to show structural details of the disclosure in more detail than is necessary for the fundamental understanding of the disclosure, the description taken with the drawings making apparent to those skilled in the art how the several forms of the disclosure can be embodied in practice.

The following definitions and explanations are meant and intended to be controlling in any future construction unless clearly and unambiguously modified in the following examples or when application of the meaning renders any construction meaningless or essentially meaningless. In cases where the construction of the term would render it meaningless or essentially meaningless, the definition should be taken from Webster's Dictionary 3^{rd} Edition.

As used herein, the term “stillage” means and refers to the feed stream.

As used herein, the term “dried distillers grains with solubles” means and refers to a co-product of dry-milled ethanol production.

As used herein, the term “polyglutamine” means and refers to a tract of at least 30 to 40 Gln amino acids encoded by CAA/CAG repeats. The polyglutamine tract can be linked and/or interrupted by other amino acids. The location of the said polyglutamine tract can be located at the beginning, the end, or within a protein.

As used herein, the term “homologous”, when used to indicate the relation between a given (recombinant) nucleic acid or polypeptide molecule and a given host organism or host cell, means and refers to the nucleic acid or polypeptide molecule is produced by a host cell or organisms of the same species, preferably of the same variety or strain. If homologous to a host cell, a nucleic acid sequence encoding a polypeptide will typically be operably linked to another promoter sequence than in its natural environment. When used to indicate the relatedness of two nucleic acid sequences the term “homologous” means that one single-stranded nucleic acid sequence can hybridize to a complementary single-stranded nucleic acid sequence. The degree of hybridization can depend on a number of factors including the amount of identity between the sequences and the hybridization conditions such as temperature and salt concentration as earlier presented. Preferably the region of identity is greater than about 5 bp, more preferably the region of identity is greater than 10 bp. Preferably, two nucleic acid or polypeptides sequences are said to be homologous when they have more than 80% identity.

As used herein, the term “heterologous”, when used with respect to a nucleic acid (DNA or RNA) or protein, means and refers to a nucleic acid or protein (also named polypeptide or enzyme) that does not occur naturally as part of the organism, cell, genome or DNA or RNA sequence in which it is present, or that is found in a cell or location or locations in the genome or DNA or RNA sequence that differ from that in which it is found in nature. Heterologous nucleic acids or proteins are not endogenous to the cell into which it is introduced, but has been obtained from another cell or synthetically or recombinantly produced. Generally, though not necessarily, such nucleic acids encode proteins that are not normally produced by the cell in which the DNA is transcribed or expressed. Similarly exogenous RNA encodes for proteins not normally expressed in the cell in which the exogenous RNA is present. Heterologous nucleic acids and proteins can also be referred to as foreign nucleic acids or proteins. Any nucleic acid or protein that one of skill in the art would recognize as heterologous or foreign to the cell in which it is expressed is herein encompassed by the term heterologous nucleic acid or protein. The term heterologous also applies to non-natural combinations of nucleic acid or amino acid sequences, i.e. combinations where at least two of the combined sequences are foreign with respect to each other.

As used herein, the term “operably linked” means and refers to a linkage of polynucleotide elements (or coding sequences or nucleic acid sequence or nucleic acid molecule) in a functional relationship. A nucleic acid sequence is “operably linked” when it is placed into a functional relationship with another nucleic acid sequence. For instance, a promoter or enhancer is operably linked to a coding sequence if it affects the transcription of the coding sequence. Operably linked means that the nucleic acid sequences being linked are typically contiguous and, where necessary to join two protein coding regions, contiguous and in reading frame.

As used herein, the term “promoter” means and refers to a nucleic acid fragment that functions to control the transcription of one or more nucleic acid molecules, located upstream with respect to the direction of transcription of the transcription initiation site of the nucleic acid molecule, and is structurally identified by the presence of a binding site for DNA-dependent RNA polymerase, transcription initiation sites and any other DNA sequences, including, but not limited to transcription factor binding sites, repressor and activator protein binding sites, and any other sequences of nucleotides known to one of skill in the art to act directly or indirectly to regulate the amount of transcription from the promoter. A “constitutive” promoter is a promoter that is active under most environmental and developmental conditions. An “inducible” promoter is a promoter that is active under environmental or developmental regulation.

As used herein, the term “marker” means and refers to a gene encoding a trait or a phenotype which permits the selection of, or the screening for, a bacterial cell containing the marker. A marker gene can be an antibiotic resistance gene whereby the appropriate antibiotic can be used to select for transformed cells from among cells that are not transformed. Preferably however, a non-antibiotic resistance marker is used, such as an auxotrophic marker (URA3, TRP1, LEU2). In a preferred embodiment, a bacterial cell transformed with a nucleic acid construct is marker gene free. Methods for constructing recombinant marker gene free microbial host cells are disclosed in EP-A-0635574 and are based on the use of bidirectional markers. Alternatively, a screenable marker such as Green Fluorescent Protein, lacZ, luciferase, chloramphenicol acetyltransferase, beta-glucuronidase can be incorporated into a nucleic acid construct of the disclosure allowing to screen for transformed cells.

As used herein, the term “Alcoli™” means and refers to a bacterium comprising a CAG/CAA repeat that encodes a polyglutamine protein. As used herein, the term “Alcoli™ Process” refers to a process for production of an organic compound utilizing the bacterium comprising a CAG/CAA repeat that encodes a polyglutamine protein.

The present disclosure relates to a scalable process for the production of an organic compound suitable as a biofuel or as chemical feedstock. The disclosure combines metabolic properties of chemoorganotrophic prokaryotes and is based on the use of recombinant heterotrophs with high rates of production of fermentative end product. In an embodiment, the novelty of the disclosure is a) that a great variety of end products can be realized by the introduction of a single nucleic acid molecule encoding a specific protein and b) that its core chemical reactions use proteins and sugars as the carbon precursor to drive the production of organic compounds. A major benefit of the process is that it converts a waste stream into ethanol.

### Escherichia Coli

In a first aspect, the disclosure provides a bacterium capable of expressing a nucleic acid molecule, wherein the expression of said nucleic acid molecule confers on the bacterium the ability increase production of a glycolytic intermediate and/or an organic compound.

In an embodiment of the disclosure, the bacterium is an Escherichia coli cell which is a heterotrophic unicellular prokaryote. This is a fast growing bacterium that can use amino acids as a carbon source. Its physiological traits are well-documented: it is able to survive and grow in a wide range of conditions.

A bacterium as defined herein is capable of increasing the production of a glycolytic intermediate and/or an organic compound as defined herein. A biochemical background of a bacterium is given in Example 1.

A bacterium as defined herein preferably comprises a nucleic acid molecule encoding a protein capable of increasing the production of glycolytic intermediates and/or an organic compound as defined herein. An organic compound is herein preferably defined as being a compound being more reduced than carbon dioxide. A bacterium is therefore capable of expressing a nucleic acid molecule as defined herein, whereby the expression of a nucleic acid molecule as defined herein confers on the bacterium the ability to increase production of glycolytic intermediates and/or an organic compound as defined herein. A glycolytic intermediate can be dihydroxyacetone-phosphate, glyceraldehyde-3-phosphate, 1,3-bis-phosphoglycerate, 2-phosphoglycerate, 3-phosphoglycerate, phospho-enol-pyruvate and pyruvate. Preferred glycolytic intermediates are pyruvate and glyceraldehyde-3-phosphate. The skilled person knows that the identity of the glycolytic intermediate converted into an organic product to be produced depends on the identity of the organic product to be produced.

Preferred organic products are selected from: a C1, C2, C3, C4, C5, or C6 alkanol, alkanediol, alkanone, alkene, or organic acid. Preferred alkanols are C2, C3 or C4 alkanols. More preferred are ethanol, propanol, and butanol. A preferred alkanediol is 1,3-propanediol. A preferred alkanone is acetone. A preferred organic acid is D-lactate. A preferred alkene is ethylene.

A preferred glycolytic intermediate for the production of ethanol, propanol, butanol, acetone or D-lactate is pyruvate. A preferred glycolytic intermediate for the production of 1,3-propanediol is glyceraldehyde-3-phosphate. A preferred glycolytic intermediate for the production of ethylene is alpha-oxyglutarate

In an embodiment, “increase production of glycolytic intermediates and/or an organic compound” can mean that detectable amounts of an organic compound are detected in the culture of a bacterium cultured for at least 1 day using a suitable assay for the organic compound.

Organic compounds produced are produced within the cell and can spontaneously diffuse into the culture broth. In an embodiment, an assay for said intermediates and alkanols, alkanones, alkanediols and organic acids is High Performance Liquid Chromatography (HPLC). A detectable amount for said glycolytic intermediates and alkanols, alkanones, alkanediols and organic acids is preferably at least 0.1 mM under said culture conditions and using said assay. Preferably, a detectable amount is at least 0.2 mM, 0.3 mM, 0.4 mM, or at least 0.5 mM.

### Organic Compound

The nucleic acid molecule codes for a protein capable of increasing pyruvate and/or an organic compound, said protein comprises a polyglutamine. In an embodiment, an assay for an organic compound is HPLC. A detectable amount of an organic compound is preferably at least 0.1 mM under said culture conditions as defined earlier herein and using said assay. In an embodiment, a bacterium comprises a nucleic acid molecule encoding a polyglutamine. In an embodiment, this relates to a bacterium capable of expressing the following nucleic acid molecule being represented by the nucleotide sequence, wherein the expression of this nucleotide sequence confers on the cell the ability to increase the production of pyruvate and/or an organic compound:

A nucleotide sequence encoding a polyglutamine, wherein said nucleotide sequence is x-CAA-x-CAG-x, in which CAA and CAG encode for glutamine, and “x” can be either CAA or CAG. Alternatively, said nucleic acid molecule and be interspersed with any codon and at any location. The polyglutamine stretch is at least 37 glutamines but can be as short as 5 glutamines or as long as thousands of glutamines. The polyglutamine tract can be located within any protein and at any location within a protein or can exist without any contiguous protein or amino acid. The CAG/CAA repeat sequence was synthesized according to Kim et al. (BioTechniques 38, 247-253).

Each nucleotide sequence encoding a protein as described herein can encode either a prokaryotic or a eukaryotic protein, i.e. a protein with an amino acid sequence that is identical to that of a protein that naturally occurs in a prokaryotic or eukaryotic organism. The ability of a protein as defined herein to confer to a bacterial cell the ability to increase the production of a glycolytic intermediate and/or an organic product does not depend so much on whether the protein is of prokaryotic or eukaryotic origin. Rather this depends on the relatedness (identity percentage) of the protein amino acid sequence or corresponding nucleotide sequence to CAA/CAG repeat sequence.

To this end, a nucleic acid construct can be constructed as described in e.g. Ordway et al., 1996, Biotechniques 21:609-612 or Chen et al., 2002, Methods in Molecular Biology, Volume 192, PCR Cloning Protocols, Humana Press, Inc. A bacterium can comprise a single but preferably comprises multiple copies of each nucleic acid construct. A nucleic acid construct can be maintained episomally and thus comprises a sequence for autonomous replication, such as an ARS sequence. Suitable episomal nucleic acid constructs can e.g. be based on the yeast 2.mu. or pKD1 (Fleer et al., 1991, Biotechnology 9:968-975) plasmids. Preferably, however, each nucleic acid construct is integrated in one or more copies into the genome of a bacterial cell. Integration into a bacterial cell's genome can occur at random by illegitimate recombination but preferably a nucleic acid construct is integrated into the bacterium cell's genome by homologous recombination as is well known in the art (U.S. Pat. No. 4,778,759). Homologous recombination occurs preferably at a neutral integration site. A neutral integration site is an integration which is not expected to be necessary for the production process of the disclosure, i.e. for the growth and/or the production of an organic compound and/or an intermediary compound as defined herein. Accordingly, in a more preferred embodiment, a bacterial cell of the disclosure comprises a nucleic acid construct comprising a nucleic acid molecule, said nucleic acid molecule being represented by a nucleotide sequence, said nucleotide sequence being a coding sequence of a protein as identified herein. Said cyanobacterial cell is capable of expression of the protein. In an even more preferred embodiment, a nucleic acid molecule encoding a protein is operably linked to a promoter that causes sufficient expression of a corresponding nucleic acid molecule in a bacterium to confer to a bacterium the ability to increase production of a glycolytic intermediate and/or an organic product. A promoter is upstream of the expressing gene. Accordingly, in a further aspect, the disclosure also encompasses a nucleic acid construct as earlier outlined herein. Preferably, a nucleic acid construct comprises a nucleic acid molecule encoding a protein as earlier defined herein. Nucleic acid molecules encoding a protein have been all earlier defined herein.

A promoter that could be used to achieve the expression of a nucleic acid molecule coding for a protein as defined herein may be not native to a nucleic acid molecule coding for a protein to be expressed, i.e. a promoter that is heterologous to the nucleic acid molecule (coding sequence) to which it is operably linked. Although a promoter preferably is heterologous to a coding sequence to which it is operably linked, it is also preferred that a promoter is homologous, i.e. endogenous to a bacterium. Preferably, a heterologous promoter (to the nucleotide sequence) is capable of producing a higher steady state level of a transcript comprising a coding sequence (or is capable of producing more transcript molecules, i.e. mRNA molecules, per unit of time) than is a promoter that is native to a coding sequence. A suitable promoter in this context includes both constitutive and inducible natural promoters as well as engineered promoters. A promoter used in a bacterium cell of the disclosure can be modified, if desired, to affect its control characteristics. In an embodiment, the promoter is a lac, lacUV5, tac, trc, trp, araBAD, phoA, recA, proU, cst-1, tetA, cadA, nar, P_{L}, cspA, SP6, T7, T7-lac operator, T3-lac operator, T5-lac operator, T4 gene 32, nprM-lac operator, VHb, or Protein A promoter.

### Method

In a second aspect, the disclosure relates to a process of increasing the production of glycolytic intermediates and/or an organic compound as defined herein by using amino acid and/or sugar as carbon sources.

A bacterium, a glycolytic intermediate, an organic compound, a nucleic acid molecule, and a regulatory system have all earlier been defined herein.

### Genetic Modifications

For overexpression of a protein in a host cell of the disclosure as described above, as well as for additional genetic modification of a host cell, preferably bacteria, host cells are transformed with the nucleic acid construct of the disclosure by methods well known in the art. Such methods are e.g. known from standard handbooks, such as Sambrook and Russel (2001) “Molecular Cloning: A Laboratory Manual (3.sup.rd edition), Cold Spring Harbor Laboratory, Cold Spring Harbor Laboratory Press, or F. Ausubel et at, eds., “Current protocols in molecular biology”, Green Publishing and Wiley Interscience, New York (1987). Methods for transformation and genetic modification of cyanobacterial cells are known from e.g. U.S. Pat. No. 6,699,696 or U.S. Pat. No. 4,778,759.

In a process of the disclosure, amino acids and sugars in the culture medium are taken in and used by the bacterium as carbon sources or proteins and carbohydrates can be broken down into amino acids and sugars inside the cells. Usually a process is started with a culture (also named culture broth) of bacteria having an optical density measured at 660 nm of approximately 0.2 to 2.0 (OD₆₆₀=0.2 to 2) as measured in any conventional spectrophotometer with a measuring path length of 1 cm. Usually the cell number in the culture doubles every 20 hours. In a preferred process, an organic compound is separated from the culture broth. This can be realized continuously with the production process or subsequently to it. Separation can be based on membrane technology and/or evaporation methods. Depending on the identity of the organic compound produced, the skilled person will know which separating method is the most appropriate.

A promoter for use in a nucleic acid construct for overexpression of a protein in a cyanobacterial cell of the disclosure has been described above. Optionally, a selectable marker can be present in a nucleic acid construct.

Optional further elements that can be present in a nucleic acid construct of the disclosure include, but are not limited to, one or more leader sequences, enhancers, integration factors, and/or reporter genes, intron sequences, centromers, telomers and/or matrix attachment (MAR) sequences. A nucleic acid construct of the disclosure can be provided in a manner known per se, which generally involves techniques such as restricting and linking nucleic acids/nucleic acid sequences, for which reference is made to the standard handbooks, such as Sambrook and Russel (2001) “Molecular Cloning: A Laboratory Manual (3rd edition), Cold Spring Harbor Laboratory, Cold Spring Harbor Laboratory Press.

Methods for inactivation and gene disruption in bacterial cells are known in the art (see e.g. Shestakov S. V. et al, (2002), Photosynthesis Research, 73: 279-284 and Nakamura Y et al, (1999), Nucleic Acids Res. 27:66-68).

### System Process for Utilization of a Bacterium Encoding a Polyglutamine Protein

In an embodiment, monitors are utilized during the glycolytic pathway. In an embodiment, the monitors are used for optimizing conditions including but not limited to pH, temperature, salt concentration, substrate levels, and cell density. In an embodiment, the monitors are used for contamination detection.

In an embodiment, there are controls for flowrate, contamination, optimization of growth, and optimization of conditions.

In an embodiment, the process uses a bacterium to convert streams containing yeast and yeast proteins into ethanol. In an embodiment, other types of cells than bacterium could be used including but not limited to algae, yeast, plant, virus, mold, and protozoa. In another embodiment, a cell and cell protein other than yeast are used including but not limited to bacteria, plant, and algae.

In this document and in its claims, the verb “to comprise” and its conjugations is used in its non-limiting sense to mean that items following the word are included, but items not specifically mentioned are not excluded. In addition the verb “to consist” can be replaced by “to consist essentially of” meaning that a peptide or a composition as defined herein can comprise additional component(s) than the ones specifically identified, said additional component(s) not altering the unique characteristic of the disclosure. In addition, reference to an element by the indefinite article “a” or “an” does not exclude the possibility that more than one of the element is present, unless the context clearly requires that there be one and only one of the elements. The indefinite article “a” or “an” thus usually means “at least one”.

All patent and literature references cited in the present specification are hereby incorporated by reference in their entirety. The following examples are offered for illustrative purposes only, and are not intended to limit the scope of the present disclosure in any way.

### EXAMPLES

### Example 1

### Biochemical Background of the Escherichia coli of the Disclosure

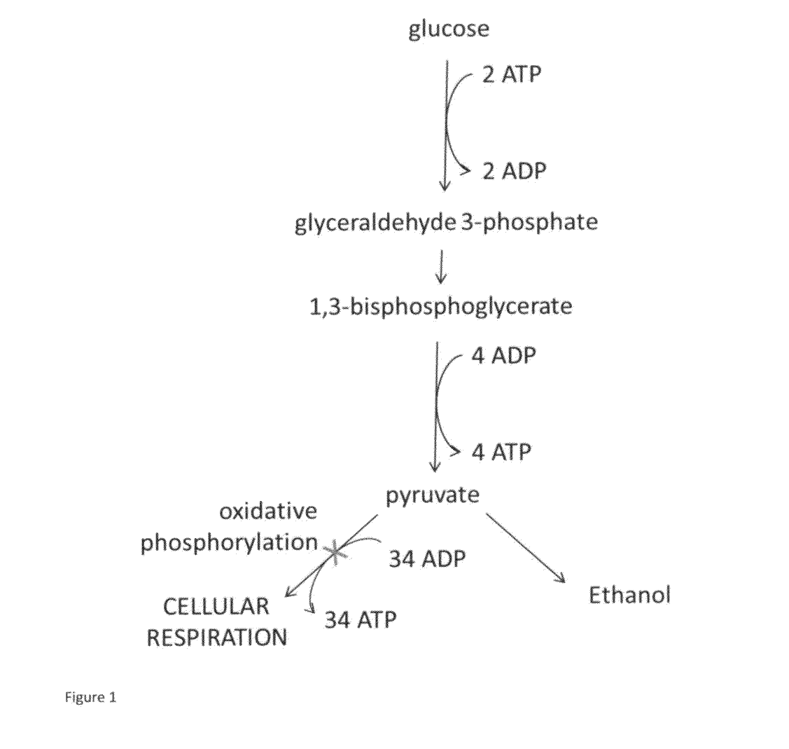

Glucose can be broken down into cellular energy (ATP) by two processes, Cellular Respiration and Fermentation. The Escherichia coli cell is engineered to shut down Cellular Respiration and, therefore, divert organic intermediate pyruvate into ethanol production (FIG. 1).

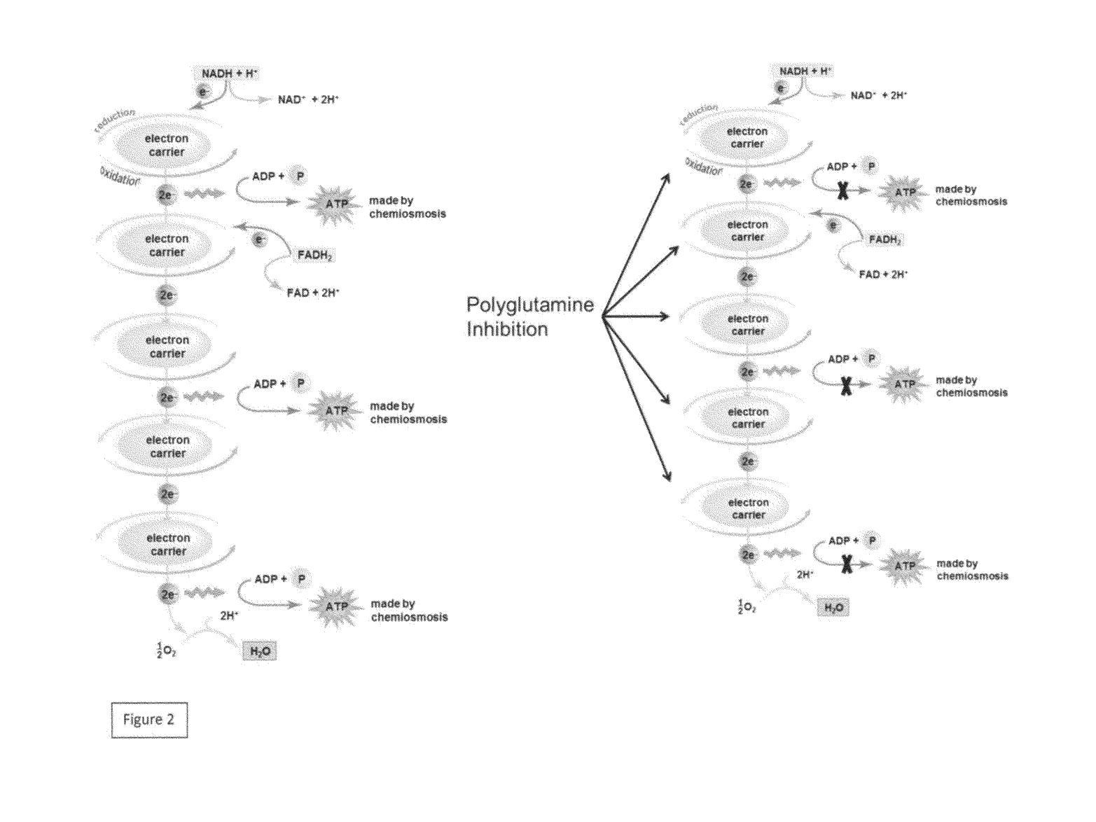

The bulk of the cellular energy ATP is made in the mitochondria during the Cellular Respiration phase of Electron Transport Chain. Polyglutamine protein has been shown to disrupt the functions of Electron Transport Chain as described by Shirendeb, U. et al. (Journal Hum. Mol. Genet. 2011 Apr. 1; 20(7):1438-55) (FIG. 2).

Cellular Respiration is the main energetic pathway in all cells. It consumes Oxygen and Glucose and yields C3 compounds (e.g. pyruvate) and ATP:

O₂+C₆H₁₂O₆→C₃ compounds+ATP (1)

Oxidative phosphorylation produces 32-34 ATP per glucose molecule, and therefore is the main phase of Cellular Respiration that sustains life through the cellular energy compound ATP.

Nature also sustains an entirely different mode of (microbial) life: Numerous bacterial and fungal species are able to conserve sufficient energy (as ATP) to proliferate by fermentation, in which they use so-called substrate level phosphorylation to generate their energy. This respiration-independent mode of energy conservation relies on metabolic pathways that result in redox neutral dissimilation of the energy source. The most abundant pathways have evolved with sugars (e.g. glucose) as energy source and therefore all have glycolysis in common:

Glucose→glyceraldehyde-P→pyruvate+reducing power (2)

Redox neutrality is maintained by the generalized reaction:

Pyruvate+reducing power→fermentation products (3)

Combining equations (2) and (3) in a redox-neutrality reaction that converts glucose into fermentation products, which are organic compounds as described herein.

### Example 2

### Description of the Expression System Used

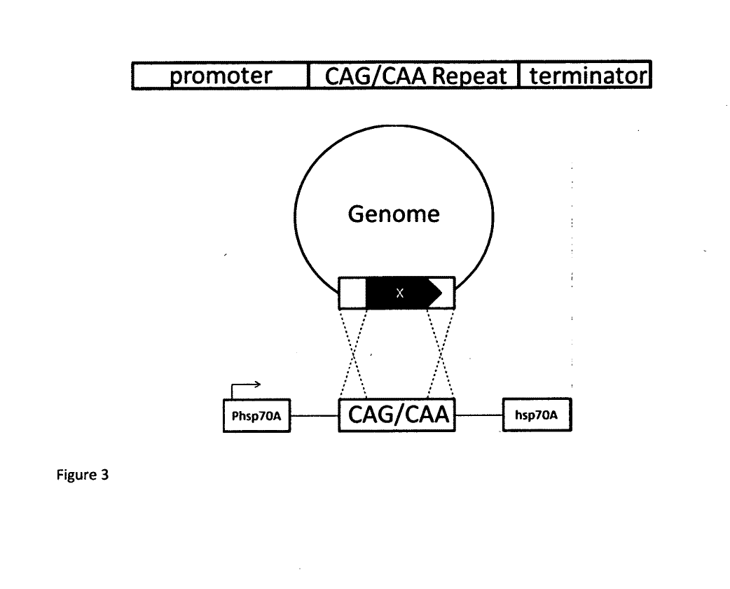

The CAG/CAA-repeat sequence is linked downstream to a transcriptional promoter such as 17 or CMV that transcribe the DNA repeat sequence into the coding mRNA sequence, which translates into the polyglutamine protein. The genetic cassette can be used to transform a cell transiently or stably by homologous recombination that incorporates the cassette into the cell's genome (FIG. 3).

### Example 3

### Amino Acids as Carbon Source

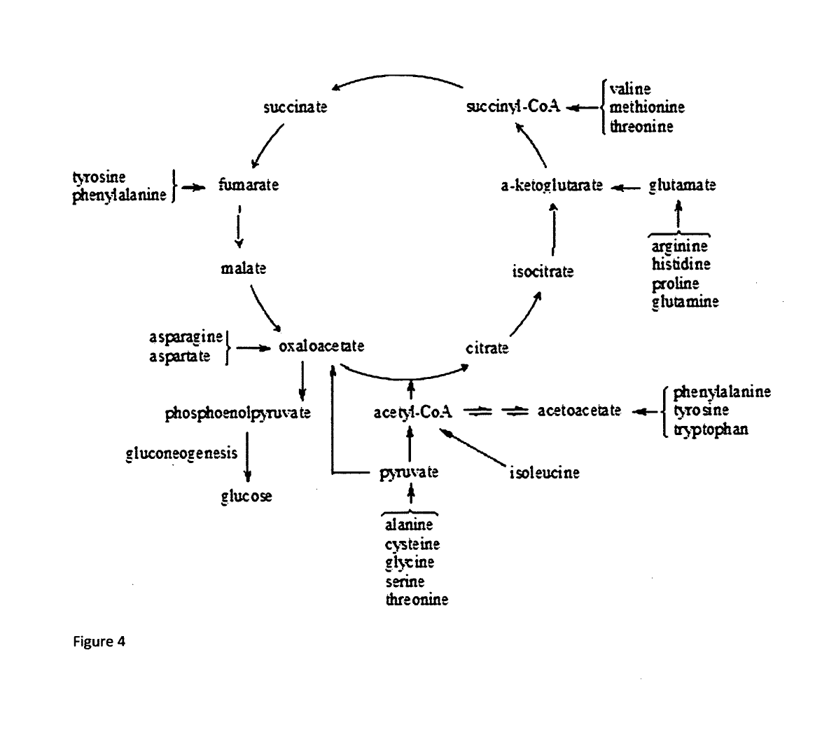

Escherichia coli is able to use amino acids as a carbon source by converting them into glucose by the biochemical process of gluconeogenesis (FIG. 4). This is described by Sezonov et al. (Journal of Bacteriology. 189(23), 8746-8749). Glucose is then used for fermentation in the engineered bacterium.

TABLE 1
List of all primers used

| | | |
|---|---|---|
| CAG-REPEAT | Forward | AATTCGAGTCGCGCG(CTG)7GCGCGACTCG |
| 1 | Reverse | GATCCGAGTCGCGC(CAG)7CGCGCGACTCG |
| ECONI 2 | Forward | GAATTCGAGTCCTGGGGGAGGACTCGGATCC |
| | Reverse | GGATCCGAGTCCTCCCCCAGGACTCGAATTC |

### Example 4

### System Process Description

The process system uses a bacterium to convert streams containing yeast and yeast proteins into ethanol. These streams are typically waste streams produced in conventional corn, sugarcane, sorghum, cassava, switchgrass, wood chips, and cellulosic feedstock based ethanol plants employing yeast in the fermentation process to produce the alcohol. The process can also be applied to yeast containing waste stream feed sources such as beverage beer, wine, bio-ethanol manufacturing, and bio-butanol manufacturing. The process can be integrated into new plant designs or retrofitted into existing facilities. In an embodiment, the conditions of the process can be changed based on the needs of the ethanol producer and location of the plant.

The process consists of essentially two steps, described in the following sections:

Feed Preparation and Fermentation

Distillation Product Recovery

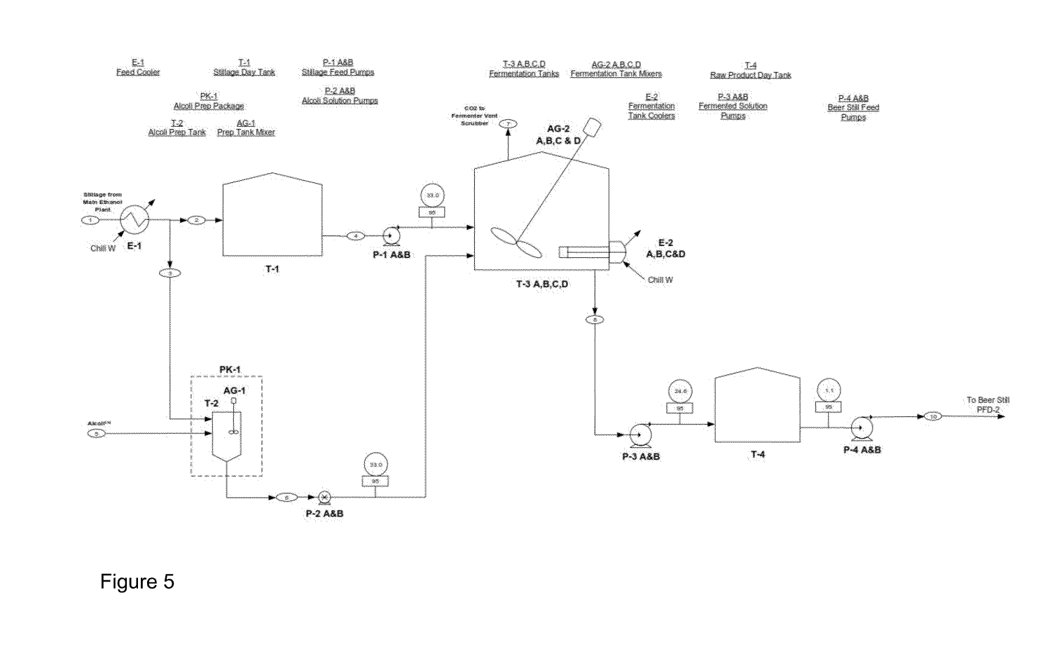

### Feed Preparation and Fermentation (FIG. 5)

Referring to FIG. 5, the feed stream (Stream 1) to the process system, often called stillage, is produced in the conventional alcohol portion of the plant. While generally coming from a separation step in the conventional plant where solids by-products are removed, depending on the conventional plant configuration, it can come from other sources within that portion of the plant.

The system bacteria are heat sensitive thus requiring controlling their use to temperatures typically lower than 100° F. Due to the stillage source conditions in the conventional the plant, the feed stream passes through Feed Cooler (E-1) heat exchanger where it is chilled typically to 95° F. The chilled stream exits the Feed Cooler after which it is split with a small portion (Stream 3) going to the Alcoli™ Prep Package Unit (PK-1) where it is mixed with Alcoli™ bacteria and in which the Alcoli™ are grown prior to being fed to the Alcoli™ system fermenters. The balance of chilled feed (Stream 2) is sent to the Stillage Day Tank (T-1). The purpose of the Stillage Day Tank is to provide a hold-up which will allow normalizing any variations in the plant feed rate. T-1 is operated at a slightly positive pressure to prevent the incursion of air. In an embodiment, the Alcoli™ (bacteria encoding a polyglutamine protein) can be added to the feed stream. In an embodiment, the feed stream can be added to the Alcoli™. In an embodiment, the Alcoli™ can be added to the feed stream inside a tank. In an embodiment, the Alcoli™ can be added to the feed stream outside of a tank. In an embodiment, the conditions within the heat exchanger can be varied. In an embodiment, the number of fermentation tanks and fermentation tank mixers can be varied.

To achieve the optimum fermentation of feed to ethanol with Alcoli™ bacteria, the fermentation is carried out, which depending on the reaction conditions, over a period of time up to 48 hours. To operate the overall process on a continuous basis the fermentation step, which is a batch operation, is conducted in several Fermentation Tanks (T-3 A, B, C & D). Each of these tanks is in a different stage of fermentation with one of the tanks always being in the state of accepting fresh feed and another of completed fermentation from which the feed to the distillation product recovery system is withdrawn.

Using the Stillage Feed Pumps (P-1 A & B) stillage (Stream 4) from the Stillage Day Tank (T-1) is pumped to the Fermentation Tank (T-3 A or B or C or D) which is operating in the fresh feed mode of the fermentation cycle. Using the Alcoli™ Solution Pumps (P-2 A & B), Alcoli™ Solution (Stream 6) also is fed to the Fermentation Tank operating in the fresh feed mode at a controlled ratio of Alcoli™ solution to stillage feed. Alternatively the Alcoli™ solution could be mixed with the Stillage Feed by being fed to either the suction or discharge side of the Stillage Feed Pumps (P-1 A & B).

In the Fermentation Tanks (T-3 A, B, C, D), the bacteria react with yeast and yeast proteins to form ethanol and carbon dioxide. Since the reaction is slightly exothermic, each tank is cooled to maintain its temperature at about 95° F. to keep the bacteria in their most active state. The cooling can be achieved either the through the use of one or more cooling type bayonet type exchangers (E-2 A, B, C, D) in each tank such as shown in PFD-1 or alternatively using in tank cooling coils or using an externally cooled pumped system. To assist in maintaining uniform reaction conditions in a tank and to assist in the cooling, each tank has one or more mixers or agitators, Fermentation Tank Mixers (AG-2 A, B, C, D) such as shown in PFD-1.

The Fermentation Tanks are operated at a slightly positive pressure. The carbon dioxide formed, saturated with water and containing a trace amount of ethanol, is sent (Stream 7) to a Carbon Dioxide Vent Scrubber which in most cases is scrubbing both this stream and the carbon dioxide vent stream coming from conventional fermenters after which it is either vented to the atmosphere or sent to a carbon dioxide recovery system.

When the ethanol content reaches about 10% (by volume), the reaction essentially ceases. At this point, the tank contents will begin to be sent, Raw Product Stream (Stream 8), to the Raw Product Day Tank (T-4) using the Fermented Solution Pumps (P-3 A & B). The fermentation tank previously sending raw product to the Raw Product Tank moves to become the fresh feed tank. The purpose of the Raw Product Day Tank is to provide a hold-up which will allow normalizing any variations in of the raw product in the fermentation step. T-4 is operated at a slightly positive pressure.

Raw Product (Stream 10) is withdrawn from the Raw Product Day Tank using the Beer Still Pumps (P4 A & B) and sent to the Distillation Product Recovery section, PFD-2, of the system.

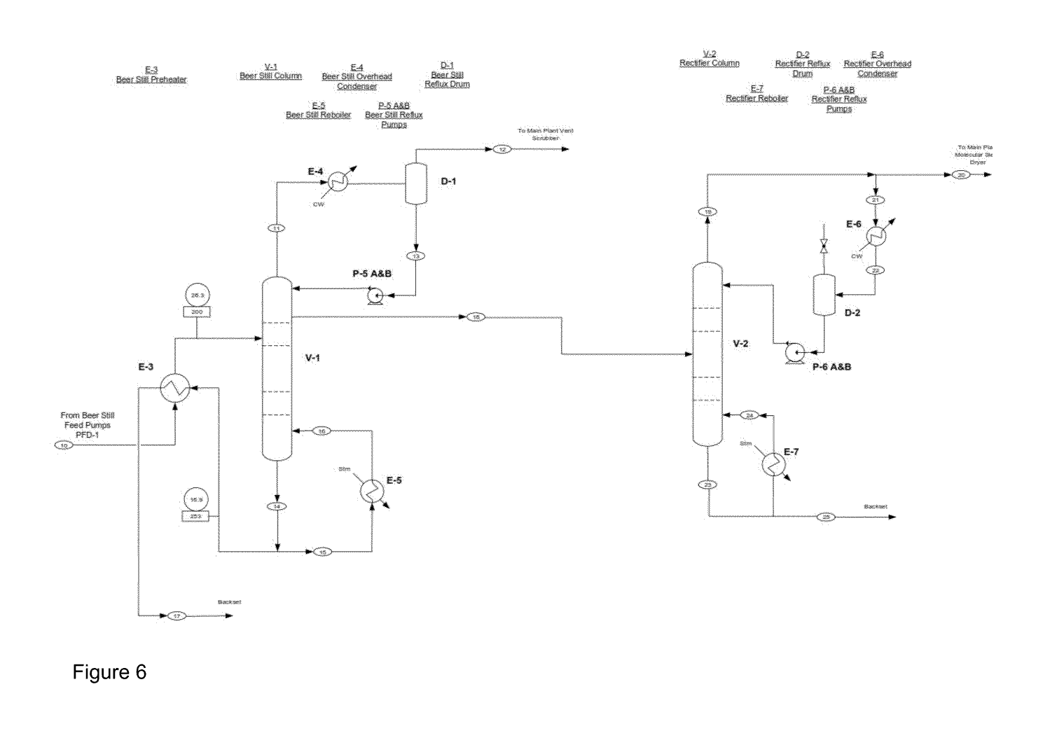

### Distillation Product Recovery (FIG. 6)

The Distillation Product Recovery system in the plant is similar in configuration to systems that are used in conventional ethanol plants to recover the ethanol product. As in conventional plants, the exact configuration and operating conditions are dependent on both the local environmental and economic conditions. As an alternative to product recovery using distillation, other separation techniques might be also used such as membrane separation or extractive distillation. A description of a typical distillation product recovery system which might be used in a plant is described in the following paragraphs.

The cool Raw Feed (Stream 10) from the Beer Still Feed Pumps (P-4 A & B) (PFD-1) is sent to Beer Still Preheater (E-3) where it is preheated by interchange with the hot Backset (Stream 17) coming from the Beer Still bottoms prior to being fed to the Beer Still Column (V-1). The Beer Still is the first of two steps designed to concentrate the product ethanol.

The Beer Still Column (V-1) is a tower in which the dilute Raw Stream is concentrated to a level of about 38% (by weight) ethanol. The heat required to achieve the separation is supplied from the Beer Still Reboiler (E-5) located at the bottom of the column. In the Beer Still Reboiler, a portion (Stream 15) of the column bottoms (Stream 14) is passes through the reboiler where a portion of the stream is vaporized. The partially vaporized stream (Stream 16) is returned to the Beer Still. The balance of the Beer Still bottoms (Stream 17) passes through the Beer Still Preheater before being sent back either to the conventional plant as backset or to waste water treatment. The Beer Still is typically a tower containing either trays or packing.

Overheads from the Beer Still Column (Stream 11) are cooled and condensed in the Beer Still Overhead Condenser (E-4) and sent to the Beer Still Reflux Drum (D-12) where non-condensable carbon dioxide along with a trace amount of ethanol and water are separated (Stream 12) and sent to the main plant vent scrubber. The condensate, consisting of ethanol and water, collected in the Beer Still Reflux Drum is refluxed to the Beer Still Column (V-1) using the Beer Still Reflux Pumps (P-5 A & B). Consisting of primarily water and ethanol the Beer Still product stream, Feed to Rectifier (Stream 18), is withdrawn from the Beer Still column and sent to the Rectifier Column (V-2).

Ethanol is further concentrated in the Rectifier Column (V-2) in which the concentration of ethanol, limited by the formation of an azeotrope of ethanol and water, reaches about 94.5% (by volume). The heat required to achieve the separation is supplied from the Rectifier Reboiler (E-7) located at the bottom of the column. In the Rectifier Reboiler, a portion (Stream 24) of the column bottoms (Stream 23) passes through the reboiler where a portion of the stream is vaporized. The partially vaporized stream (Stream 24) is returned to the Rectifier Column. The balance of the Rectifier Column bottoms (Stream 25) is sent back either to the conventional plant as backset or to waste water treatment. The Rectifier Column is typically a tower containing either trays or packing.

The overhead vapor from the Rectifier Column (Stream 19) is split with part of the flow (Stream 21) being returned as reflux to the Rectifier Column with balance being the product ethanol rich stream, Hydrous Ethanol (Stream 20). Prior to being returned to the Rectifier Column the reflux stream is cooled and condensed in Rectifier Overhead Condenser and then sent (Stream 22) to the Rectifier Reflux Drum (D-2) where trace non-condensables e.g. carbon dioxide are removed and relieved as needed to the main plant vent scrubber. The condensate, consisting of ethanol and water, collected in the Rectifier Reflux Drum is refluxed to the Rectifier Column (V-2) using the Rectifier Reflux Pumps (P-6 A & B). Consisting of primarily water and ethanol the Beer Still product stream, Feed to Rectifier (Stream 18), is withdrawn from the Beer Still column and sent to the Rectifier Column (V-2).

The product Hydrous Ethanol (Stream) containing about 94.5% (by volume) of ethanol is sent to main plant molecular sieve drying system or a separate molecular sieve drying system in the unit in which the remaining water is essentially removed.

### Material Balance Summary

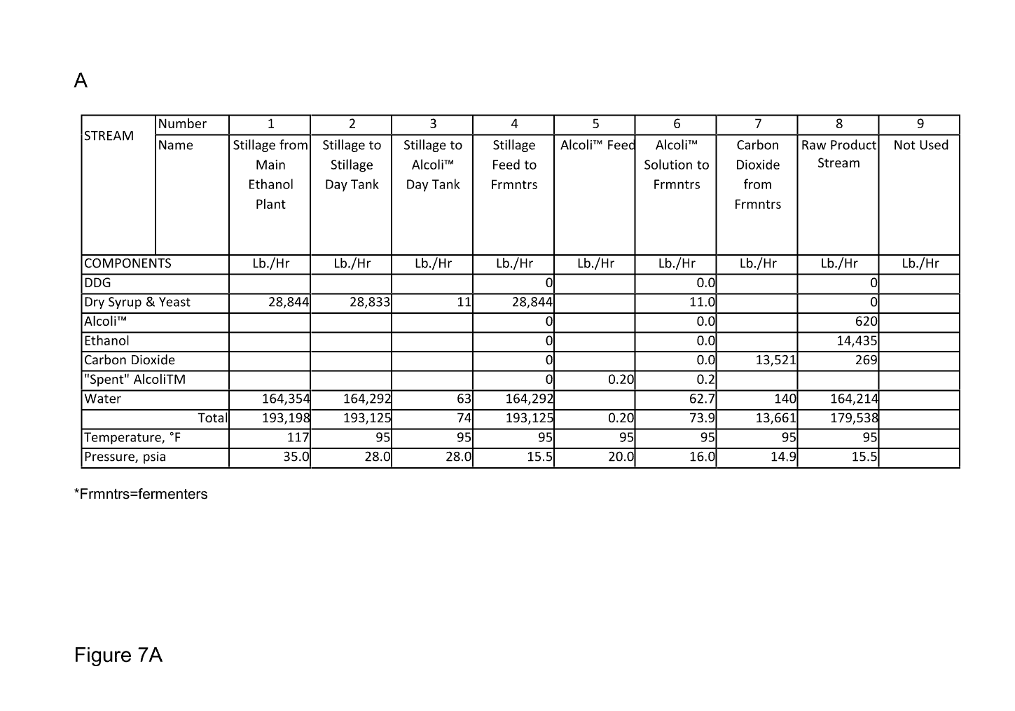

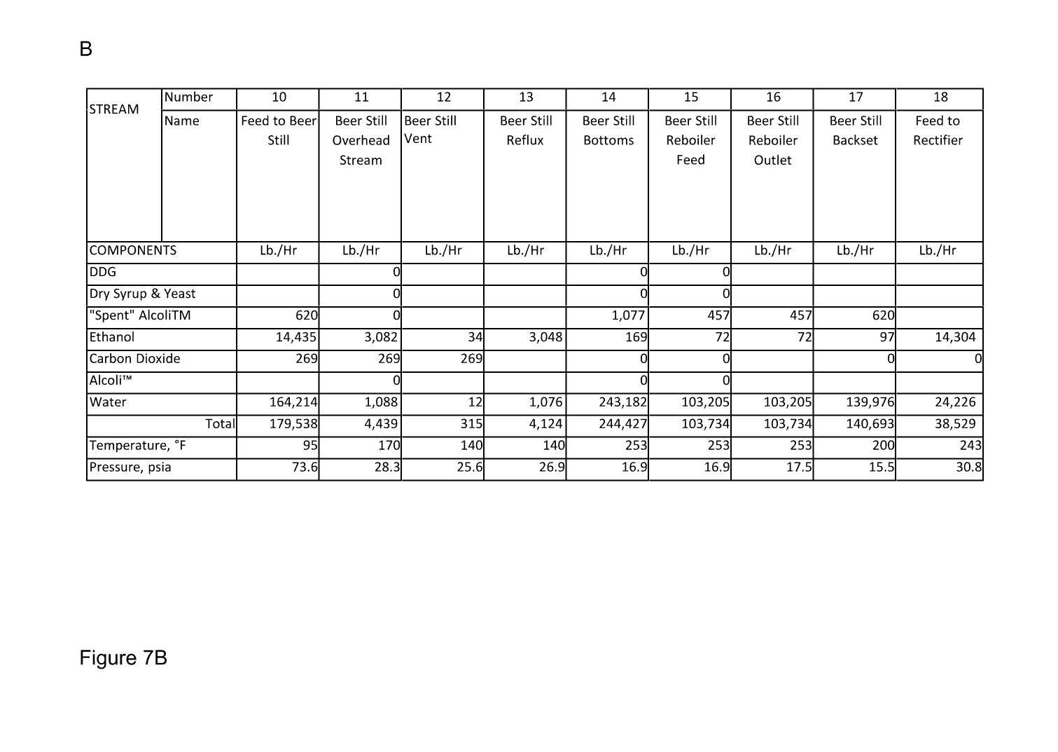

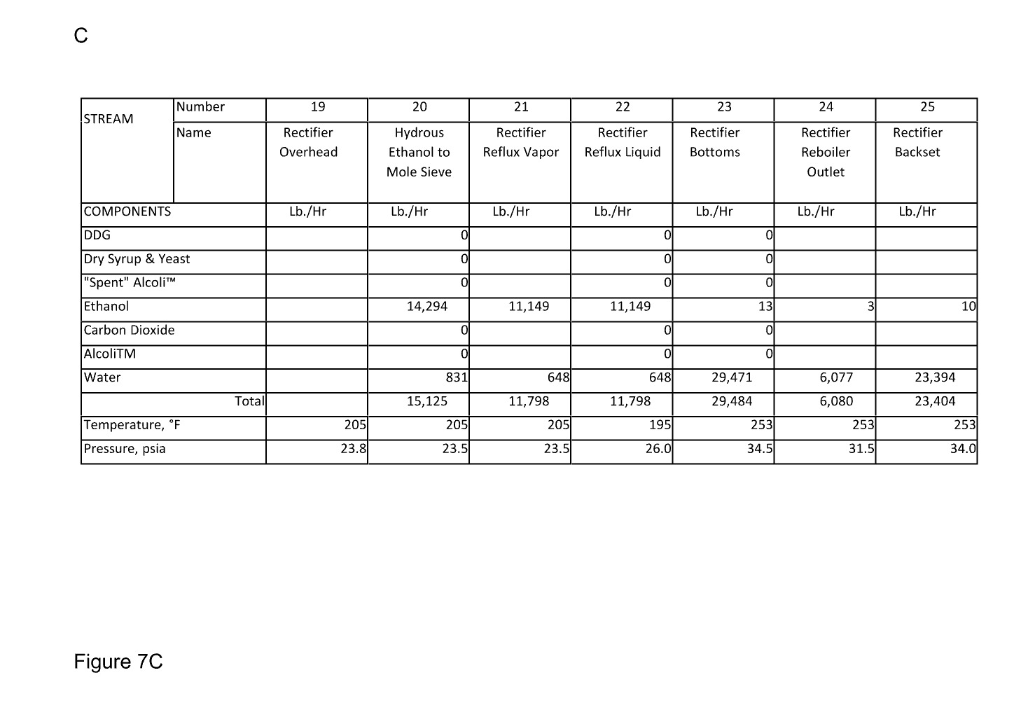

FIG. 7A-7C depict a mass balanced summary for Streams 1-25.

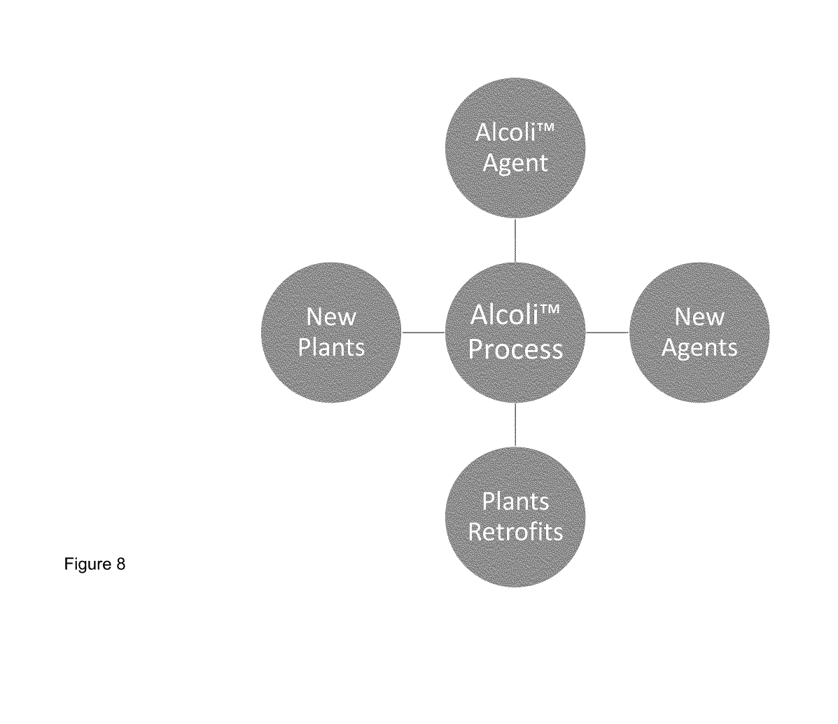

In an embodiment, the new agents are yeast, bacteria, algae, plants, molds, and protists. (FIG. 8).

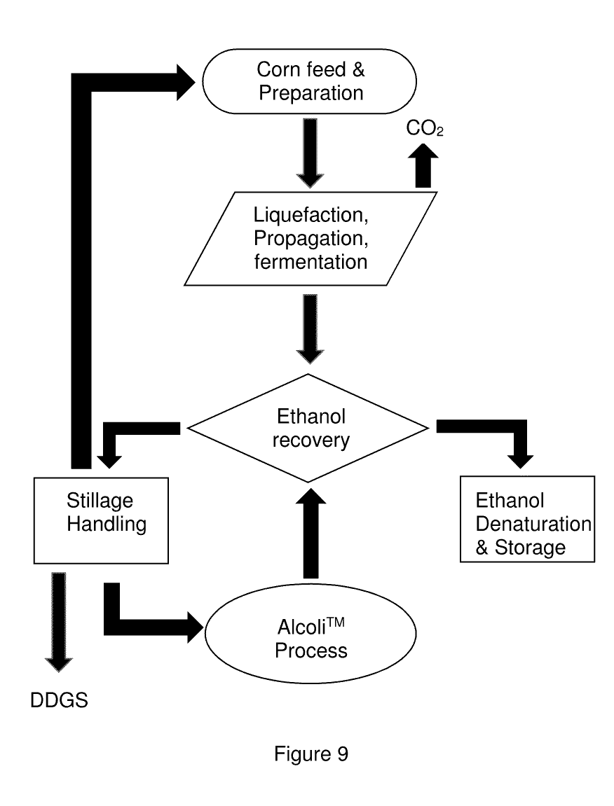

FIG. 9 depicts the interaction of ethanol production from corn feed and the Alcoli™ process. In an embodiment, the process can allow for a similar yield to the ethanol plant alone but with the use of less feed stock, such as corn, sugarcane, sorghum, cassava, switchgrass, and wood chips.

In an embodiment, the conditions, used during production of the organic compound such as temperature and pressure, can be varied. In an embodiment, the fermentation temperature range is 39° F. to 160° F. In an embodiment, the fermentation temperature is 95° F. In an embodiment, the pressure range is from below at atmospheric pressure to about 30 psia. In an embodiment, the pressure range is from about 20 psia to about 30 psia.

All of the compositions and methods disclosed and claimed herein can be made and executed without undue experimentation in light of the present disclosure. While the compositions and methods of this disclosure have been described in terms of preferred embodiments, it will be apparent to those of skill in the art that variations can be applied to the compositions and methods and in the steps or in the sequence of steps of the methods described herein without departing from the concept, spirit and scope of the disclosure. More specifically, it will be apparent that certain agents which are both chemically related can be substituted for the agents described herein while the same or similar results would be achieved. All such similar substitutes and modifications apparent to those skilled in the art are deemed to be within the spirit, scope and concept of the disclosure as defined by the appended claims.

### REFERENCES

Kim S H., Cai L., Pytlos M., Edwards S., Sinden R. (2005) Generation of long tracts of disease-associated DNA repeats. BioTechniques 38, 247-253.
Hongmin W., Lim P., Karbowski M., Monteiro M. (2009) Effects of overexpression of Huntingtin proteins on mitochondrial integrity. Hum. Mol. Gen. 18(4), 737-752.
Sezonov G., Jose-Petit D., D'Ari R. (2007) Escherichia coli physiology in Luria-Bertani broth. Journal of Bacteriology. 189(23), 8746-8749.
Dale, Rhys T. and Tyner, Wallace E., Staff Paper #06-04, Economic and Technical Analysis of Ethanol Dry Milling, Agricultural Economics Department, Purdue Univ., April 2006.
Humbird, D. Davis, R, Tao, L, Kinchin, C., Hsu, D., Aden, A., Schoen, P., Lukas, J., Olthof, B., Worley, M., Sexton, D., and Dudgeon, D., Process Design and Economics for Biochemical Conversion of Lignocellulosic Biomass to Ethanol, Dilute-Acid Pretreatment and Enzymatic Hydrolysis of Corn Stover, U.S. Department of Energy—National Renewable Energy Laboratory, May 2011.
Maria et al., 2001, The Modification of Protein Metabolism of Sunflower Plants Under Saline Stress. Romanian Agricultural Research 16, 5-14, 2001.
Asishet al., 2005, High salinity reduces the content of a highly abundant 23-kDa protein of the mangrove Bruguiera parviflora. Planta 221, 135-140.
Kemp et al., 1972, Protein Metabolism in Cultured Plant Tissues. Plant Physiology 49, 596-601.
Menegus et al., 1993, Rice and wheat seedlings as plant models of high and low tolerance to anoxia. In: Hochachka P, ed. Surviving hypoxia: mechanisms of adaptation and control. Boca Raton: CRC Press, 53-64.
Baily-Serres, 1990, Hypoxic stress induced changes in ribosomes of maize seedling roots. Plant Physiology 94, 1237-1243.
Sachs et al., 1996, Anaerobic gene expression and flooding tolerance in maize. Journal of Experimental Botany 47, 1-15.
Varapetian et al., 1978, Plant cells under oxygen stress. In: Hook D D, Crawford R M M, eds. Plant life in anaerobic environments, 13-88, Ann Arbor: Ann Arbor Science, 13-88.
Miyuki et al., 2002, The α-Amylase Induction in Endosperm during Rice Seed Germination Is Caused by Gibberellin Synthesized in Epithelium. Plant Physiology, 128 (4), 1264-1270. EP-A-0635574.
Kim et al., 2005, “Generation of long tracts of disease-associated DNA repeats” BioTechniques 38, 247-253.
Ordway et al., 1996, “In vitro synthesis and cloning of long CAG repeats” Biotechniques 21:609-612.
Chen et al., 2002, Methods in Molecular Biology, Volume 192, PCR Cloning Protocols, Humana Press, Inc.
Fleer et al., 1991, Biotechnology 9:968-975.
U.S. Pat. No. 4,778,759.
Sambrook and Russel (2001) “Molecular Cloning: A Laboratory Manual (3.sup.rd edition), Cold Spring Harbor Laboratory, Cold Spring Harbor Laboratory Press.
F. Ausubel et at, eds., “Current protocols in molecular biology”, Green Publishing and Wiley Interscience, New York (1987).
U.S. Pat. No. 6,699,696.
U.S. Pat. No. 4,778,759.
Shestakov S. V. et al, (2002), Photosynthesis Research, 73: 279-284.
Nakamura Y et al, (1999), Nucleic Acids Res. 27:66-68.
Shirendeb, U. et al., Journal Hum. Mol. Genet. 2011 Apr. 1; 20(7):1438-55.
R. Katzen, P. Madson & G. Moon, Jr., Chapter 18: Ethanol distillation: the fundamentals, The Alcohol Textbook: A reference for the beverage, fuel and industrial alcohol industries by Jacques, Lyons and Kelsall. Nottingham University Press (2nd Edition) 1995, Third edition 1999.

What is claimed is:

1. A process for increasing the production of a glycolytic intermediate and the production of an organic compound by bacteria comprising
preparing a feed stream;
combining the bacteria and feed stream;
fermenting the feed stream;
cooling the feed stream during fermentation;
recovering the organic compound; and
concentrating the organic compound;
wherein the bacteria comprises a nucleic acid molecule comprising a CAG/CAA repeat that encodes a polyglutamine protein that increases production of a glycolytic intermediate; and
wherein a starting substrate is selected from at least one of the group consisting of sugar and protein.

2. The process of claim 1 wherein the organic compound is selected from the group consisting of ethanol, butanol, ethylene, 1,3-propanediol, propanol, D-lactate, acetone, and fatty acid.

3. The process of claim 1 wherein the glycolytic intermediate is pyruvate.

4. The process of claim 1 wherein the bacteria is Escherichia coli.

5. The process of claim 1 wherein the feed stream comprises yeast cells or portions thereof.

6. The process of claim 5 wherein the feed stream is a waste stream.

7. The process of claim 6 wherein the waste stream is produced from one selected from the group consisting of corn, sugarcane, sorghum, cassava, switchgrass, and wood chips.

8. The process of claim 6 wherein the waste stream is produced from one selected from the group consisting of beer, wine and bio-butanol manufacturing.

9. The process of claim 1 wherein the polyglutamine protein is that of SEQ ID NO: 1.

10. The process of claim 1 wherein preparing the feed stream comprises lowering the temperature of the feed stream.

11. The process of claim 1 wherein recovering the organic compound comprises utilizing one method selected from the group consisting of distillation, membrane separation, and extractive distillation.

12. The process of claim 1 further comprising concentrating the organic compound by at least one column.

13. The process of claim 1 further comprising removing at least some of the water from the ethanol.

14. The process of claim 13, wherein removing at least some of the water from the ethanol by a molecular sieve drying system.

15. The process of claim 1 wherein the nucleic acid molecule comprising a CAG/CAA repeat that encodes a polyglutamine protein is integrated into the genome of the bacteria.

16. The process of claim 1 wherein the nucleic acid molecule comprising a CAG/CAA repeat that encodes a polyglutamine protein is present on a plasmid.

17. The process of claim 1 wherein the organic compound comprises about 94.5% ethanol.

18. The process of claim 1 wherein fermenting the feed stream occurs at a positive pressure.

19. The process of claim 18 wherein the positive pressure is between about 20 psia and 30 psia.

20. The process of claim 1 wherein the temperature during fermenting the feed stream is about 95° F.
